(19) United States
(12) Patent Application Publication
(10) Pub. No.: US 2026/0283523 A1
(43) Pub. Date: Sep. 24, 2026

(54) STRETCHABLE MICRONEEDLE ELECTRODE ARRAYS AND GEL-ASSISTED PATTERNING OF THREE-DIMENSIONAL STRUCTURES

(71) Applicant: University of Southern California, Los Angeles, CA (US)

(72) Inventors: Hangbo ZHAO, Los Angeles, CA (US); Qinai ZHAO, Los Angeles, CA (US)

(21) Appl. No.: 19/475,253

(22) PCT Filed: May 24, 2024
(86) PCT No.: PCT/US2024/031147

§ 371 Date: Oct. 14, 2025

Related U.S. Application Data
(60) Provisional application No. 63/468,959, filed on May 25, 2023.
(60) Provisional application No. 63/469,024, filed on May 25, 2023.

(51) Int. Cl. A61B 5/262; A61B 5/251; A61B 5/268; A61B 5/296

(52) U.S. Cl. CPC A61B 5/262; A61B 5/251; A61B 5/268; A61B 5/296

(57) ABSTRACT

A microneedle electrode array is disclosed. The microneedle electrode array can include a substrate configured to stretch or move between a first state and a second state. A plurality of microneedles can extend outwardly from the substrate and include a base. A plurality of electrical interconnects is extended from the plurality of microneedles.

### CROSS REFERENCE TO RELATED APPLICATIONS

This application claims priority to U.S. Provisional Appl. No. 63/469,024, filed on May 25, 2023, and to U.S. Provisional Appl. No. 63/468,959, filed on May 25, 2023, each of which is herein incorporated by reference in their entirety.

### STATEMENT AS TO FEDERALLY SPONSORED RESEARCH

This invention was made with government support under grant nos. N0014-19-1-2373 and N00014-21-1-2927, awarded by the Office of Naval Research (ONR). The government has certain rights in the invention.

### TECHNICAL FIELD

Disclosed herein are systems, devices, and/or methods relating to microelectrode arrays for biomedical applications and, more particularly, the design, fabrication, and electrophysiological applications of highly stretchable microneedle electrode array devices. Disclosed herein are also systems, devices, and/or methods relating to patterning of three-dimensional structure.

### BACKGROUND

Microelectrode arrays (MEA) have been a widely used technological platform for biomedical applications including electrophysiological recording, electrochemical sensing, and electrical stimulation. Conventional MEAs have planar electrode layouts fabricated on rigid substrates (e.g., silicon and glass) using lithographical processes, such as one-dimensional (1D) Michigan probe for neural recording, and two-dimensional (2D) MEAs on glass for cell culture studies. Out-of-plane 2D MEAs, such as the Utah array, expand the electrode-bio interface to the third dimension. Moreover, 3D MEAs that allow electrode coverage in 3D space are achievable by stacking 1D or 2D MEAs, 3D printing, or complex nanofabrication techniques such as focused ion beam (FIB) milling. 3D MEAs can penetrate surfaces of tissues, thereby allowing sensing of physiological signals and electrical stimulation of the interior or deep tissues in a minimally invasive manner. Such multi-channel, deep tissue sensing, and stimulation capabilities can provide valuable information and control over a wide range of 3D biological systems such as the brain tissues, skin dermis layers, neuromuscular tissues, skeletal tissues, and organoids and assembloids.

In addition to achieving high electrode density in 3D space, advances in flexible or stretchable MEAs can mitigate the mechanical mismatch between rigid MEAs with soft, curvilinear, and dynamic biological tissues. Flexible 3D MEAs are accessible through multiple fabrication schemes, including wet etching and release of silicon, replica molding, magnetorheological drawing, and controlled bending of 2D MEAs. While flexible 3D MEAs provide improved conformability to irregular or curvilinear surfaces, the stretchability of these devices is highly desirable due to their capability of following deformations of dynamically moving tissues or organs such as the brain, the heart, and musculature systems. This dynamic conformability of 3D MEAs to tissues could allow stable bioelectronic interfacing, enhanced recording signal quality, and reduced tissue damage, all of which are important for in vivo applications.

However, stretchable 3D MEAs present significant fabrication challenges, mainly due to the incompatibility of existing rigid, penetrating electrode fabrication processes and the stretchability requirements for the constituent materials and structures. Current stretchable 3D MEAs have limitations in fabrication scalability and cost, as well as device stretchability. Examples include stainless steel microneedle electrode arrays assembled manually on a silicone substrate, and silicon-based microneedle electrodes fabricated by complex etching with approximately 20% maximum stretching.

Further, the patterning of 3D micro/nanoscale structures is an important step in fabricating various functional micro- and nanostructures and systems, including biosensors, tissue scaffolds, drug delivery systems, optical/optoelectronic devices, energy conversion and storage systems, microelectromechanical systems (MEMS), and self-cleaning surfaces. The fabrication of some of these structures and devices involves patterning functional materials on protruding structures such as micro- and nanoscale pillars, needles, and wires. These protruding micro/nano structures can be important components in functional devices like electrodes for sensing, stimulation, and energy storage, probes for biochemical sensing and reactions, and active sites for cell/tissue attachments, and the like.

Advances in planar microfabrication methods have enabled micro- and nanoscale patterning of a wide range of materials, typically through lithography and pattern transfer processes such as material deposition and etching. Additional planar patterning techniques have also been developed, including contact-based printing methods like screen printing, gravure printing, and flexographic printing, as well as non-contact-based methods such as inkjet printing, electrohydrodynamic printing, and laser ablation. However, a significant limitation of these planar patterning techniques is that they are generally inapplicable or poorly suited to non-planar surfaces.

Patterning on 3D surfaces at the micro- and nanoscale has been challenging, requiring unconventional material deposition or removal processes with high spatial resolution in three dimensions. One example of such a technique is material deposition and ablation using a focused ion beam (FIB), where a fine beam of high-energy ions acts as a direct-write tool to create patterns or remove materials on a 3D surface with high precision. However, this process requires expensive equipment and has slow processing speeds. Advanced 3D printing techniques such as two-photon polymerization and aerosol jet printing can achieve micro- and nanoscale resolution for material deposition, but limitations exist in material compatibility and their applicability to existing curved surfaces.

This disclosure resolves these and other issues of the art.

### SUMMARY

The subject of this disclosure includes microneedle electrode array that can include a substrate able to stretch or move between a first state and a second state. The microneedle electrode array can include a plurality of microneedles extending outwardly from the substrate and including a base. A plurality of electrical interconnects can be extended from the plurality of microneedles.

In some examples, the base of the plurality of microneedles is bonded to the substrate.

In some examples, the plurality of electrical interconnects can be connected to the plurality of microneedles. In some examples, the plurality of electrical interconnects and the plurality of microneedles are bonded to the substrate.

In some examples, the substrate is formed from silicone, and the microneedles and the electrical contacts are covalently bonded to the substrate.

In some examples, the plurality of microneedles has a conical shape extending axially from the base to a tip.

In some examples, the electrical interconnects have a serpentine pattern extending along a surface of the substrate.

In some examples, the electrical interconnects are formed from a polymeric material having an electrically conductive material disposed on an outer surface.

In some examples, each of the electrical interconnects is individually connected to a respective microneedle and an external circuit.

In some examples, the microneedles are formed from a polymeric material having an electrically conductive material disposed on an outer surface.

In some examples, a tip of the microneedles includes electrochemical deposition of a coating including a conductive material that decreases an average electrode impedance of the microneedle electrode array to less than or equal to approximately 2.3 kΩ at 1 kHz in 0.1 M phosphate-buffered saline (PBS).

In some examples, the microneedle electrode array includes an electrode impedance that is relatively unchanged below approximately 40% strain.

In some examples, the microneedle electrode array includes an electrode impedance that significantly increases from approximately 50-70% strain.

In some examples, the microneedle electrode array includes an electrode impedance that significantly increases from approximately 60-90% strain.

In some examples, a method is disclosed related to making a microneedle electrode array. The method can include forming a plurality of cavities having a microneedle shape on a surface of a mold substrate; filling the plurality of cavities and the surface of the mold substrate with a polymeric material to form a plurality of microneedles; patterning the polymeric material on the surface of the mold substrate to form a pattern of a plurality of interconnects on the surface of the mold substrate that are connected with and extended from the plurality of microneedles; attaching the pattern of the plurality of interconnects and the plurality of microneedles to an array substrate; removing the mold substrate to expose the plurality of microneedles and the pattern of the plurality of interconnects as attached to the array substrate; and disposing an electrically conductive material onto an exposed surface of the plurality of microneedles and the pattern of the plurality of interconnects.

In some examples, the method can include removing polymeric material in excess of the plurality of microneedles and the plurality of interconnects from the elastomeric substrate.

In some examples, the step of forming is performed by laser micromachining process.

In some examples, before the step of attaching, the method includes treating the pattern of the plurality of interconnects and the plurality of microneedles to form a chemical bond with the array substrate.

In some examples, the array substrate includes a silicone material and the chemical bond formed with the plurality of interconnects and the plurality of microneedles is a covalent bond.

In some examples, during the step of patterning, the plurality of interconnects is formed having a serpentine pattern along the surface of the mold substrate to enable the plurality of interconnects to move and maintain contact with a respective microneedle of the plurality of microneedles with the array substrate stretching between a first state and a second state.

In some examples, during the step of forming and/or filling, the microneedles include a conical shape including a base attached to the array substrate and a tip that is positioned an axial distance from the base.

In some examples, the step of disposing the electrically conductive material onto the exposed surface of the plurality of microneedles includes electrochemical deposition of a thin layer of a conductive coating layer onto the exposed surface.

In some examples, the step of disposing the electrically conductive material onto the exposed surface of the plurality of microneedles at least partially causes an average electrode impedance to decrease to less than or equal to approximately 2.3 kΩ at 1 KHz in 0.1 M PBS.

In some examples, a method is disclosed related to treating a three-dimensional structure. The method can include moving a surface portion of the three-dimensional structure into a deformable material; treating the surface portion disposed into the deformable material by removing material from the surface portion or applying material onto the surface portion; and removing the treated surface of the three-dimensional structure from the deformable material.

In some examples, the three-dimensional structure is protruding micro/nano structure.

In some examples, the three-dimensional structure is one or more protruding microneedles.

In some examples, the surface to be treated is an end portion of the three-dimensional structure.

In some examples, the surface portion to be treated is defined by the depth of the end portion inserted into the deformable material.

In some examples, the deformable material is a gel.

In some examples, the gel includes a material disposed therein configured to remove material from the surface.

In some examples, wherein the gel includes a material disposed therein is configured to deposit a material onto the surface during the step of treating.

In some examples, the gel includes an etchant material to remove material from the surface during the treating step.

In some examples, the gel is porous.

In some examples, the step of moving includes using an optical device to control contact with or insertion of the surface into the deformable material.

In some examples, the three-dimensional structure is a protruding micro/nano structure including a plurality of end surfaces to be treated.

In some examples, during the step of moving, the plurality of end surfaces is disposed within the deformable material, and wherein during the step of treating, the plurality of end surfaces is simultaneously treated by contact with the chemical in the deformable material.

In some examples, the step of treating the surface portion disposed into the deformable material by removing material from the surface portion or applying material onto the surface portion includes material etching at least partially in an inserted region of the deformable material.

In some examples, the step of treating the surface portion disposed into the deformable material by removing material from the surface portion or applying material onto the surface portion includes material deposition at least partially in an inserted region of the deformable material.

In some examples, a method is disclosed related to use of any microneedle electrode array of this disclosure to record electrophysiological signals within dynamically moving tissues, such as intramuscular electromyography (EMG). In some examples, the method can include implanting the microneedle electrode array in a region of a patient (e.g., a region of the patient's heart) and recording electrophysiological signals related to the region of the patient's heart.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A depicts a stretchable microneedle electrode array according on an example embodiment of this disclosure.

FIG. 1B depicts the stretchable microneedle electrode array of FIG. 1A after being stretched to a second state according on an example embodiment of this disclosure.

FIG. 2A is a close-up view of Section 2A of FIG. 1A.

FIG. 2B is a close-up view of Section 2B of FIG. 1B.

FIGS. 3-5 show an example schematic illustration of steps for fabricating example arrays of this disclosure.

Views A to D of FIG. 6 show schematic illustrations related to one example of controlling the recording length of a microneedle with a conductive tip according to aspects of this disclosure.

Views A to E of FIG. 7 show aspects of an example stretchable microneedle electrode arrays for electrophysiological sensing.

Views A to I of FIG. 8 show schematic illustrations related to example implementations with related experimental data according to aspects of this disclosure.

FIG. 9 shows schematic illustrations related to example implementations with related experimental data according to aspects of this disclosure.

Views A to C of FIG. 10 show schematic illustrations related to example implementations with related experimental data according to aspects of this disclosure.

FIG. 11A shows a side-view optical image of a PDMS mold with conical cavities fabricated by laser micromachining according to aspects of this disclosure.

FIG. 11B shows a side-view of optical images of example fabricated microneedles according to aspects of this disclosure.

FIG. 12A shows side-view optical images of example microneedles with different base diameters and heights on an example substrate according to aspects of this disclosure.

FIG. 12B shows an optical image of example microneedles with PI interconnects on a silicone substrate at an angle-view of a microneedle array at scale bars of 5 mm according to aspects of this disclosure.

FIG. 12C shows an optical image of example microneedles with PI interconnects on a silicone substrate at an angle-view of a microneedle array at scale bars of 500 μm according to aspects of this disclosure.

FIG. 13 shows layouts of an example PI pattern (subviews (i)) and an example sacrificial layer pattern (subviews (ii)) for a row of microneedle electrodes with view A at 1 mm spacing and view B at 2 mm spacing according to aspects of this disclosure.

FIG. 14 shows layouts of an example PI pattern (view A) and an example sacrificial layer pattern (view B) for an example array according to aspects of this disclosure.

FIG. 15A shows SEM images of a microneedle tip (View A) before and (View B) after etching an example hard mask at the tip according to aspects of this disclosure.

FIG. 15B shows an optical image of a Cu-coated microneedle tip immersed in the custom gel etchant according to aspects of this disclosure.

FIG. 16A shows a SEM image of a microneedle tip after electrochemical deposition at the tip according to aspects of this disclosure.

FIG. 16B shows electrode impedance spectra and average electrode impedance at 1-kHz scanning frequency before and after electrochemical deposition at the microneedle tip in 0.1 M phosphate-buffered saline (PBS) according to aspects of this disclosure.

FIG. 16C shows impedance of the electrode with the microneedle tip at 1 KHz under cyclic insertion into agarose gel according to aspects of this disclosure.

FIG. 17A shows a SEM image of an example microneedle with the tip exposed with a scale bar of 200 μm according to aspects of this disclosure.

FIG. 17B shows an optical image of an example patterned gel etchant for etching a hard mask on a microneedle in an array of microneedles with varying heights with a scale bar of 300 μm according to aspects of this disclosure.

FIG. 18A shows an example pattern of thin film for tensile testing according to aspects of this disclosure.

FIG. 18B shows a stress-strain curve of the thin film example of FIG. 20B from uniaxial tensile testing.

Views A to F of FIG. 19 show schematic illustrations related to example implementations of microneedles according to aspects of this disclosure.

FIG. 20A shows finite element analysis (FEA) results related to the equivalent strain distribution in a metal layer for deformations shown previously in view A of FIG. 10.

FIG. 20B shows FEA results related to the equivalent strain distribution in a metal layer for deformations shown previously in view A of FIG. 10.

Views A to C of FIG. 21 show schematic illustrations with related experimental data according to aspects of this disclosure.

FIG. 22 illustrates a flowchart for a method, according to an embodiment.

FIG. 23 illustrates a flowchart for a method, according to an embodiment.

### DETAILED DESCRIPTION

Disclosed herein are systems, devices, and methods relating to stretchable 3D MEAs that can be constructed in a manner that are configured for scalable fabrication, increased electrode modulus, varying electrode lengths, controlled recording areas and electrode impedance, and increased device stretchability when compared to conventional stretchable 3D MEAs. Disclosed herein are also systems, devices, and/or methods that enable low-cost, scalable, high-resolution patterning techniques for 3D micro- and nanoscale structures.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise.

In this disclosure, the term “comprising” is synonymous with “including,” “having,” “containing,” or “characterized by.” These terms are inclusive and open-ended and do not exclude additional, unrecited elements or method steps. By using any of these terms, it is meant that at least the named compound, element, particle, or method step is present in the composition or article or method, but does not exclude the presence of other compounds, materials, particles, method steps, even if the other such compounds, material, particles, method steps have the same function as what is named.

In this disclosure, the phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

In this disclosure, the phrase “consisting essentially of” limits the scope of a claim to the specified materials or steps, plus those that do not materially affect the basic and novel characteristic(s) of the claimed subject matter.

In this disclosure, where publications are referenced, the disclosures of these publications in their entireties are hereby incorporated by reference into this application.

In this disclosure, relative terms, such as “about,” “substantially,” or “approximately” are used to indicate a possible variation of ±10% in the stated value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

The following examples illustrate the various embodiments of the present disclosure. Those skilled in the art will recognize many variations that are within the spirit of the present disclosure and scope of the claims.

Disclosed herein are highly stretchable microneedle electrode array devices (SMNEA), methods for making the same. In some aspects, the herein disclosed system and related processes provide a low-cost and scalable microneedle electrode fabrication process that can combine laser micromachining, replica molding, microfabrication, and transfer printing allows the formation of individually addressable, high-modulus microneedle arrays connected to interconnects. In some aspects, the solutions of this disclosure have been specially developed and engineered to enable scalable fabrication while having relatively high electrode modulus (e.g., E=6.6 GPa). In some aspects, the solutions of this disclosure facilitate use with varying electrode lengths (e.g., provide precise control over the exposed electrical recording regions, regardless of the microneedle length), provide controlled recording areas, electrode impedance, and have relative improved device stretchability (e.g., approximately 50 to 60 percent, approximately 60 to 90 percent, etc.).

Systems, devices, and methods are also disclosed that use porous, liquid-filled gels to achieve conformal contact with protruding micro/nano structures. In an example, the liquid or particles inside the liquid are contained within a porous gel. Upon partial insertion of protruding micro/nano structures into the gel, physical or chemical processes such as material etching or nanoparticle deposition occur at the gel-structure interfaces. Due to the liquid-absorbing properties of the gel, the etching or deposition processes can be confined to the contact areas between the protruding structures and the gel. In some aspects, the contact area can be controlled by adjusting the insertion location and depth, which can be guided by optical imaging. In this respect, the fabrication of microneedle electrodes with patterned tips for localized electrical recording has been demonstrated, using liquid gel (e.g., etchant-filled agarose gel).

In some aspects, the exposed lengths on the microneedle electrodes can be controlled to be within approximately 10 μm as compared to the set lengths. In some aspects, a patterning step using liquid etchant-filled gel can be convenient, low-cost, and scalable. Moreover, such gel patterning method as disclosed herein allows for controlled tip exposure for microneedles with different lengths, readily achievable by shaping the gel etchants, e.g., into narrow cubes, for the separate insertion of individual microneedles. Controlled etching at the tips for microneedles is also possible with vision-based, automated control of insertion, given the short insertion time, or by creating gel etchant patterns with predefined heights.

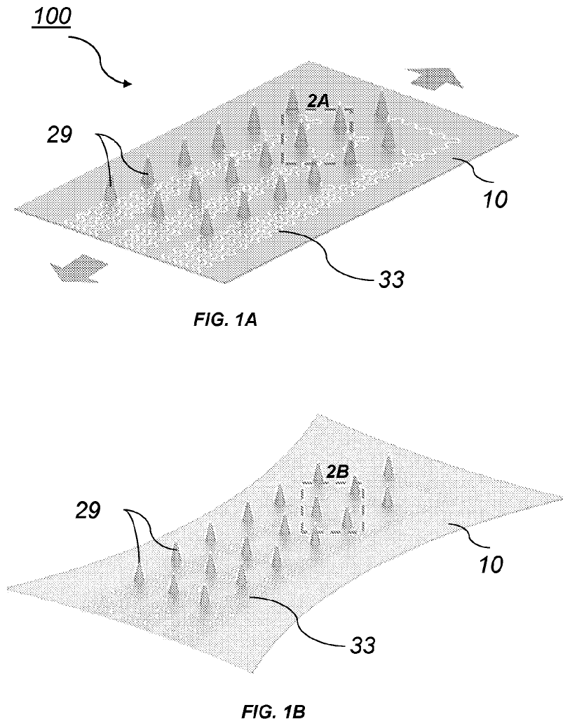

Turning to FIG. 1A and FIG. 1B, an example microneedle electrode array system 100 is shown. System 100 can include a substrate 10 configured to stretch or move between a first state (before stretching as in FIG. 1A) and a second state (after at least partial uniaxial stretching, as in FIG. 1B).

In some aspects, for applications of system 100 in electrophysiological recordings, control of the recording regions is typically necessary to target the regions of interest. This can be realized by conformal coverage of a thin insulation layer (e.g., parylene C) on microneedles 29 except for the tip 29a. This can serve as the exposed conductive regions of microneedles 29 for electrophysiological recording. In this respect, system 100 can be configured for electrophysiological sensing, such as on human subjects inside or outside the body. In some aspects, the system 100 can be used on a location of an organ (e.g., one or more regions of the heart such as, but not limited, to the aorta, one or more arteries, one or more ventricles, etc.). In this respect, in some aspects the system 100 can be used to record electrophysiological signals within dynamically moving tissues, such as intramuscular EMG. In some examples, the system 100 can be implanted in a region of a patient (e.g., a region of the patient's heart) and recording electrophysiological signals, using electrodes of the system 100, related to the region of the patient's heart.

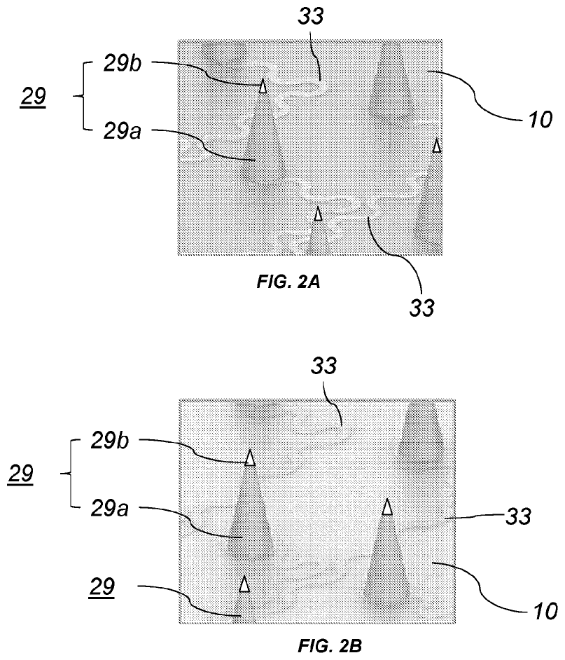

Substrate 10 can include an elastomer (e.g., an elastomer that includes silicone such as Ecoflex 00-30). System 100 can include a plurality of microneedles 29 extending outwardly from substrate 10. As shown more clearly in FIGS. 2A and 2B, each of which are close-up views of section 2A of FIG. 1A and section 2B of FIG. 1B, each microneedle 29 can include a base 29a that extends from substrate 10. In some aspects, the microneedles 29 can be formed from a polymeric material having an electrically conductive material disposed on an outer surface. In some aspects, base 29a can include an insulation coating. In one example, microneedles 29 can be made of polyimide (e.g., PI 2610) with a conductive coating (e.g., Cr/Au of approximately 10 nm/150 nm in thickness) and an insulating coating of parylene C (5 μm in thickness). In some aspects, base 29a can be bonded or otherwise attached to substrate 10. Microneedles 29 can be any number of shapes, including but not limited to a conical shape extending axially from base 29a to a tip 29b. In some aspects, tip 29b can be conductive. Microneedles 29 can have base diameters ranging from approximately 300 to 800 μm, lengths ranging from approximately 500 to 2000 μm, and tip diameters of approximately 10 to 30 μm.

System 100 can include a plurality of electrical interconnects 33 electrically connected and extended from microneedles 29. In some aspects, interconnects 33 can include a serpentine pattern, as in FIGS. 1A to 2B, extending along a surface of the substrate 10 (e.g., the illustrated visible upper surface of substrate 10). Interconnects 33 can be connected to microneedles 29 by being bonded or otherwise attached to substrate 10. In some aspects, microneedles 29 and corresponding electrical contacts can be covalently bonded to substrate 10. In one example, interconnects 33 can include polyimide filaments (e.g., approximately 15 μm in thickness and approximately 70 μm in width) with a relatively thin metal film (e.g., Au of approximately 150 nm). In some aspects, filaments of each of the interconnects 33 are individually connected to a respective microneedle 29 and an external circuit. In some aspects, upon stretching of substrate 10, as seen between FIGS. 1A and 1B, interconnects 33 bonded to substrate 10 can accommodate the deformation through in-plane bending of the example arc-shaped patterns. It is understood that other patterns that are not necessarily arc-shaped are contemplated which can still result in accommodation of deformation through in-plane bending. In some aspects, covalent bonding between microneedles 29 with interconnects 33 and substrate 10 can prevent delamination between them.

This disclosure is more clearly understood with corresponding studies discussed more particularly below. It is understood that data is presented herein for purposes of illustration and should not be construed as limiting the scope of the disclosed technology in any way or excluding any alternative or additional embodiments.

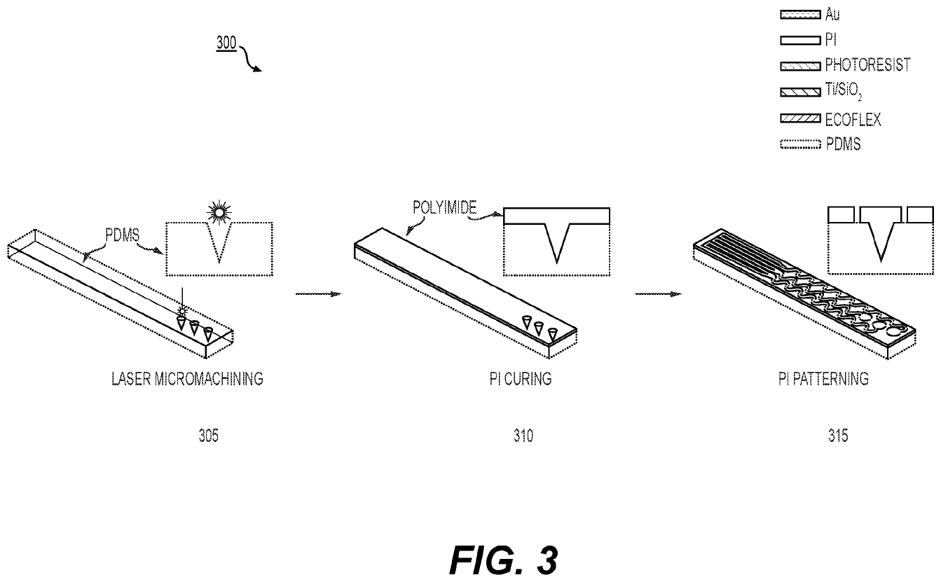

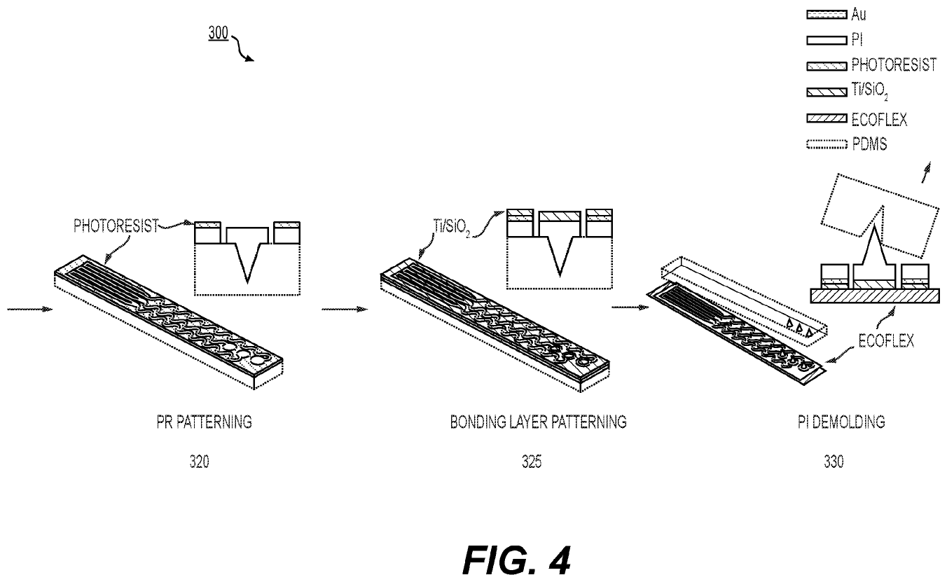

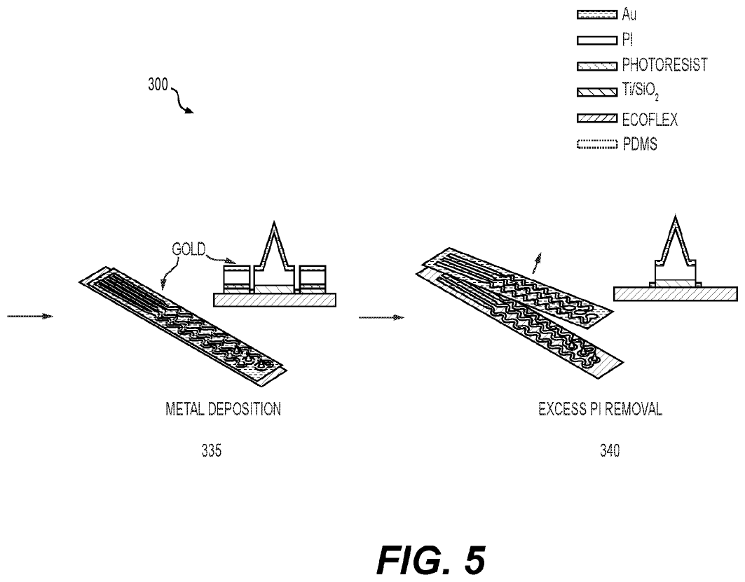

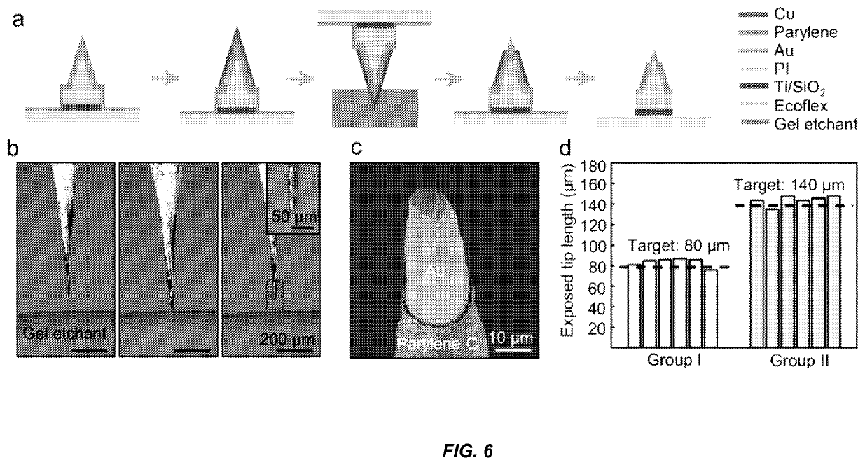

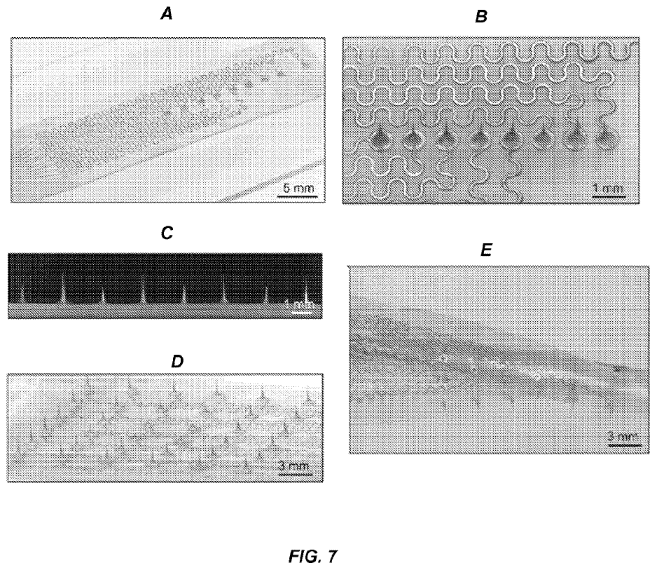

In one example, FIGS. 3-5 show a schematic illustration of steps in process 300 for fabricating example arrays of this disclosure. Process 300 can be configured to provide metallization of microneedles 29 and interconnects 33 of system 100 to create conductive pathways from microneedle surfaces to external aspects, such as an external circuit, external electronics, etc. For sensing or stimulation applications that utilize example systems of this discourse (e.g., system 100), control of the active bioelectronic interfacing region is important to target the regions of interest which can be achieved through process 300. In the depicted aspects of FIG. 3, process 300 can include a laser micromachining step 305, a PI curing step 310, and a PI patterning step 314. In the depicted aspects of FIG. 4, process 300 continues and can include a PR patterning step 320, a bonding layer patterning step 325, and a PI demolding step 330. In the depicted aspects of FIG. 5, process 300 continues and can include a metal deposition step 335 and an excess PI removal step 340.

In process 300, it was observed that indentions of microneedles with similar geometries (e.g., approximately 920 μm in length, approximately 10 to 30 μm in tip diameter, etc.) against flat rigid surfaces provide critical buckling loads. It was observed that systems fabricated using process 300, including their respective microneedles, resulted in force-displacement curves of a relatively low Young's modulus PI (PI-2545, E=2.3 GPa) and a relatively high Young's modulus PI (PI-2610, E=6.6 GPa) during indentation tests. An average critical buckling load was observed as approximately 73 and approximately 139 mN for PI-2545 and PI-2610 microneedles, respectively. In some aspects, a simulated critical buckling load was observed to depend on certain boundary conditions. The critical buckling load of microneedles produced via process 300 with a pinned tip was observed as approximately 3.6 times greater than that with a free tip. The experimentally measured buckling loads for both PI-2545 and PI-2610 microneedles were observed to fall between the simulated values for free tip and pinned tip boundary conditions.

Insertion tests of microneedles into PDMS (10:1 mixing ratio) were observed to provide additional comparison of microneedles with different moduli. The force-displacement curve during insertion was observed to yield the insertion force, defined as the load at the inflection point where penetration of the microneedle tips in the PDMS occurs. PI-2545 microneedles were observed to exhibit insertion forces in the range of approximately 44 to 66 mN, which was close to their critical buckling loads from the indentation tests (e.g., approximately 67 to 80 mN). Subsequent increases in the resisting force after insertion lead to buckling and bending of the microneedles. In contrast, the PI-2610 microneedles not only exhibited larger critical buckling loads but also reduced insertion forces (e.g., approximately 25 to 30 mN). In some aspects, as the critical buckling loads are approximately four to five times higher than the insertion forces, PI-2610 microneedles can be inserted into the PDMS without buckling.

To achieve selective insulation, some approaches have included a sacrificial coating followed by etch back, selective etching of the insulation coating by microneedles punching through a film, mechanical tearing of the insulation coating, insulation of the microneedle bases through gravity-driven flow or spin coating, and bulk silicon etching. Yet, each of these approaches either lacks reliable, precise control of the exposed areas or requires complicated fabrication processes. Another common limitation is that none of these techniques applies to an array of microneedles with varying lengths. The stretchability of system 100 brings more challenges to this problem. To address these challenges, process 300 provides convenient access to precisely controlling the electrical recording areas of system 100, which applies to microneedles with varying lengths, such as microneedles 29 of this disclosure.

Views A to D of FIG. 6 show schematic illustrations related to an example of controlling the recording length of a microneedle with a conductive tip according to aspects of this disclosure. In particular, the schematic illustration of an example fabrication process of conductive systems, such as system 100, appears in view A of FIG. 6. This hybrid fabrication strategy allows the formation of highly customized microneedle geometries and array layouts using low-cost laser ablation and standard microfabrication processes. View A of FIG. 6 shows a schematic illustration of steps for fabricating an example microneedle electrode (e.g., microneedle 29) with the conductive tip (e.g., tip 29b) exposed. View B of FIG. 6 shows side-view microscopic images of etching of a copper hard mask at the tip 29b by inserting the tip 29b in a gel etchant, where scale bars are 200 μm. View C of FIG. 6 shows an example SEM image of a tip 29b after etching the example parylene coating at the tip, where scale bars are 10 μm. View D of FIG. 6 shows statistics of exposed tip lengths from two groups of microneedles with target exposed tip lengths of 80 μm and 140 μm, respectively.

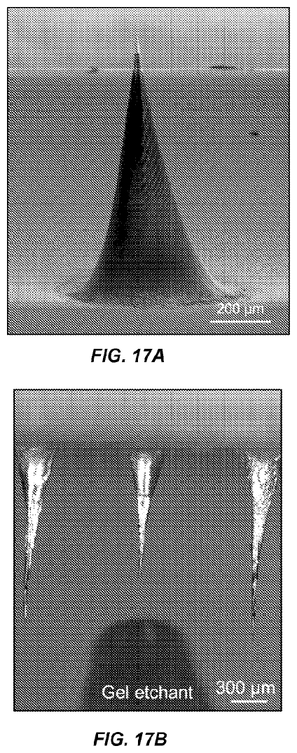

A key step in creating a well-defined recording area is the use of a gel etchant that can etch the Cu coating at the tips of the microneedles. The gel etchant is formed by soaking 0.6% agarose gel in a mixture of FeCl₃/HCl solution to allow diffusion of FeCl₃/HCl into the gel. A brief (e.g., approximately 5 s) insertion of the tip of the Cu-coated microneedle in the gel etchant allows the dissolution of the Cu film on the inserted tip, followed by removal of the remaining Cu hard mask using liquid FeCl₃/HCl (example SEM images appear in FIG. 17A).

In some aspects, the length of the etched Cu at the tip can be controlled by controlling the insertion depth of the microneedle, which is controllable by a motorized stage with optical imaging (see view B of FIG. 6). With the Cu hard mask removed at the tip, an oxygen plasma etching of the parylene coating exposes the Au layer at the tip (see view C of FIG. 6). The use of a gel etchant instead of a liquid-phase FeCl₃/HCl was observed to minimize the spreading of the liquid etchants along the microneedle due to liquid wetting on the Cu surface. The etchant spreading distance observed was approximately 20 μm when a gel etchant was used (see FIG. 17B), in contrast to complete spreading of etchant onto the microneedle surfaces when using liquid-phase FeCl₃/HCl solution. In some aspects, the measured exposed tip lengths were approximately 81.5±5.5 μm and approximately 141.5±6.5 μm for target lengths of 80 μm (group I) and 140 μm (group II), respectively (see view D of FIG. 6). FIG. 19A shows the example SEM image of an example microneedle electrode with the tip exposed using the method described here.

In some aspects, the gel etching method of this disclosure is configured to provide controlled tip exposure for microneedles with different lengths, which is readily achievable by shaping the gel etchant into narrow cubes for the insertion of individual microneedles separately (see, e.g., FIG. 19B discussed below). Controlled etching at the tips for a large number of microneedles is also possible with vision-based, automated control of insertion given the short insertion time, or by creating gel etchant patterns with predefined aspects (e.g., predetermined heights).

Large arrays of stretchable microneedle electrodes with varying microneedle lengths, such as system 100, can be created using this hybrid fabrication approach, as shown in views A to D of FIG. 7. Views A and B of FIG. 7 show optical images of example fabricated systems (e.g., system 100) laminated on a glass substrate at low and high magnification. Each of views A and B show the microneedle electrodes (e.g., microneedles 29) with interconnects (e.g., interconnects 33). Scale bars are 5 mm in view A and 1 mm in view B. View C of FIG. 7 shows a side-view optical image of a microelectrode array with varying microneedle lengths, ranging from approximately 800-1500 μm, where the scale bars are 1 mm. View D of FIG. 7 shows angled-view optical image of an example 6×6 array of microelectrodes with varying length on a stretchable silicone substrate. View E of FIG. 7 shows an example optical image of a 6×6 array of microelectrodes under stretching and twisting. Scale bars are 3 mm in view D and in view E.

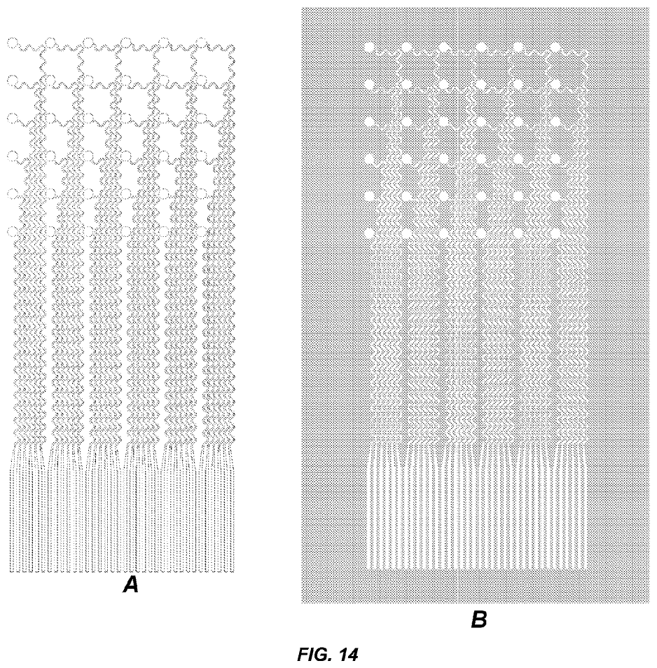

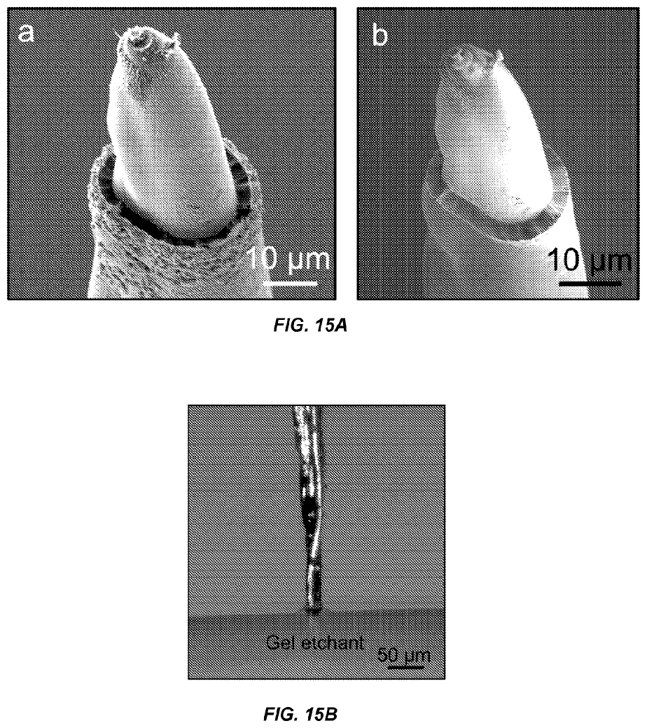

The process of FIG. 6 can start with a conformal coating of parylene C (3 μm in thickness) on the microneedles and the Ecoflex substrate, followed by patterned deposition of a hard Cu mask through a shadow mask (view A of FIG. 6) that prevents Cu deposition outside of the microneedle surfaces. A key step in creating a well-defined recording area is the use of a gel etchant that can etch the Cu coating at the tips of the microneedles. The gel etchant can be formed by soaking 0.6% agarose gel in a mixture of FeCl₃/HCl solution to allow diffusion of FeCl₃/HCl into the gel. A brief (e.g., approximately 5 s) insertion of the tip of the Cu-coated microneedle in the gel etchant can allow the dissolution of the Cu film on the tip followed by removal of the remaining Cu hard mask using liquid FeCl₃/HCl. Example SEM images related to this process appear in views A and B of FIG. 15A of an example microneedle tip (a) before and (b) after etching the Cu hard mask at the tip. Scale bars in FIG. 15A are 10 μm in a and 100 μm in b. The use of a gel etchant instead of a liquid-phase FeCl₃/HCl was observed to minimize the spreading of the liquid etchants along the microneedle due to liquid wetting on the Cu surface. The etchant spreading distance is approximately 20 μm when a gel etchant is used, as in FIG. 15B, in contrast to complete spreading of etchant onto the microneedle surfaces when using liquid-phase FeCl₃/HCl solution.

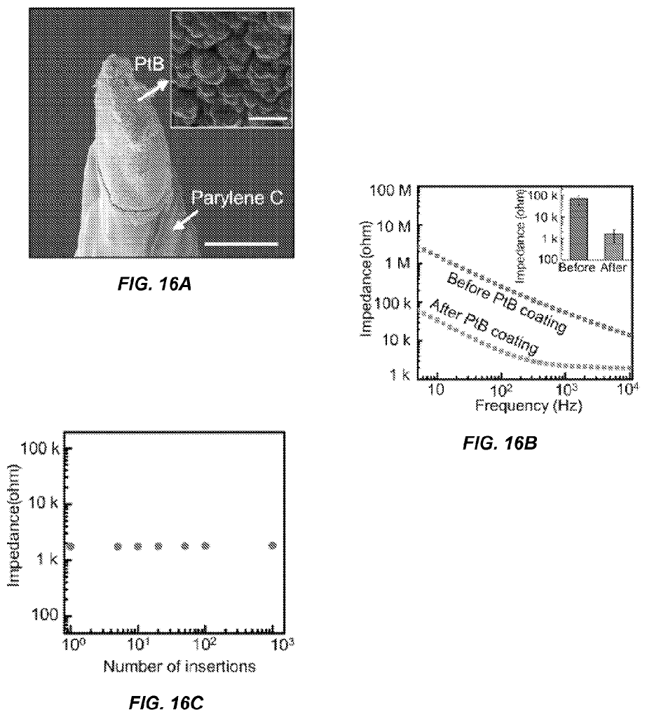

The electrical impedance of the microneedle electrodes with exposed Au tip (approximately 80 μm exposed length) appears in FIG. 16B. An additional coating of a nanoporous conductive layer, such as platinum black (PtB), on the exposed Au, was observed to decrease the electrical impedance of the microneedle electrode by increasing the electrode surface area. FIG. 16A shows an SEM image of a microneedle tip after the electrochemical deposition of a thin layer of PtB coating (e.g., approximately 1 μm in thickness), which was observed to decrease the average electrode impedance measured in PBS from 53 kΩ to 2.3 kΩ at 1 KHz scanning frequency (FIG. 16B). In other similar test of the system, was observed to decrease the average electrode impedance measured in PBS from 66.2 to 1.6 kΩ at 1 kHz scanning frequency. It was observed that this the PtB coating provided control over the electrode impedance without changing the recording areas.

In some aspects, for applications of system 100 in electrophysiological recordings, control of the recording regions is typically necessary to target the regions of interest. This can be realized by conformal coverage of a thin insulation layer (e.g., parylene C) on microneedles 29 except for the tip 29a. This can serve as the exposed conductive regions of microneedles 29 for electrophysiological recording. One approach contemplated for a selective insulation strategy can include a sacrificial coating followed by etch back, selective etching of the insulation coating by microneedles 29 punching through a film, mechanical tearing of the insulation coating, insulation of the microneedle bases through gravity-driven flow or spin coating, and bulk silicon etching. Yet, each of these approaches either lacks reliable, precise control of the exposed areas or requires complicated fabrication processes. A common limitation is that none of these techniques applies to an array of microneedles with varying lengths. The stretchability of system 100 brings more challenges to this problem. To address these challenges, FIG. 6A provides convenient access to precisely controlling the electrical recording areas of system 100, which applies to microneedles 29 with varying lengths.

In some aspects, one example fabrication process can begin with a step that includes preparing gels capable of absorbing and retaining liquids due to their porous structures, formed by a network of interconnected polymer chains. In some aspects, hydrogels represent an important category of such gels, with strong water-absorbing capacity, including agar, polyacrylamide, alginate, and gelatin. The gel can be soaked in liquid or colloidal solutions. The liquid used may have a wide range of compositions for various patterning applications and may include insoluble nanoparticles in the form of a colloid.

The table below shows some examples of constituents in the liquid/colloid contemplate for use in material deposition and/or etching processes:

| Potential constituents | | |
|---|---|---|
| in liquid/colloid | Examples of chemicals | Example applications |
| Solvents | Water, organic solvents such | Dissolving materials |
| | as methanol, | |
| | and isopropanol | |
| Acids | Hydrochloric acid (HCl), | Etching metals or metal oxides |
| | sulfuric acid (H₂SO₄), acetic | |
| | acid (CH₃COOH) | |
| Base | Potassium hydroxide, sodium | Etching silicon or glass |
| | hydroxide | |
| Oxidizing agents | Hydrogen peroxide (H₂O₂), | Converting material into a |
| | potassium permanganate | more soluble form |
| | (KMnO₄), and potassium | |
| | iodate (KIO₃) | |
| Surfactants | Polyvinyl alcohol (PVA), | Modifying wetting properties |
| | Triton X-100, and | of liquids |
| | cetyltrimethylammonium | |
| | bromide (CTAB) | |
| Inhibitors | Benzotriazole (BTA) | Slowing down the etching rate |
| | | of certain materials or to |
| | | protect |
| | | specific areas of the surface |
| Nanoparticles | Metals, metal oxides, | Deposition of nanoparticles for |
| | semiconductor nanoparticles, | various applications |
| | carbon nanotubes (CNTs), | |
| | graphene, graphene oxide, | |
| | magnetic nanoparticles, | |
| | ceramic nanoparticles, | |
| | polymer nanoparticles | |

During the above soaking process, liquid or colloidal solutions can be absorbed by the gels with micro/nanoscale pores. The soaking time can depend on the porosity of the gels and the interfacial properties of the liquids and gels. After the soaking process, the gel can be used for material etching or deposition through contact with target structures. In some aspects, protruding structures can be brought into contact with the gel and pushed to penetrate the gel under precision control. Penetration of the protruding structures into the gel allows conformal contact between the liquid-filled gel and non-planar surfaces of the protruding structures. Maintaining the contact allows the constituents in the liquid to interact with the structure surfaces, which could initiate one or more of dissolving or etching of materials on the target surface; deposition of functional nanoparticles in the colloidal solutions absorbed by the gel; and other physical or chemical processes that alter the surface properties of the target surface, such as modifications of the surface wettability of the target surface.

In some aspects, penetration location and depth can be controlled by motion stages directed by optical imaging methods such as optical microscopy for microscale patterning and more advanced electron microscopy for nanoscale patterning. After a certain amount of interaction time, the gel and the target structure can be separated, which can end the patterning process. During the contact of the gel and the target surface, the liquid (with potential colloidal particles inside) absorbed in the gel contacts the target surface, with limited liquid spreading out from the contact area due to the liquid retaining properties of the porous gel. As a result, the material etching or deposition can occur mainly in the inserted region of the surfaces of the target structure. This is in contrast to printing or wet etching processes where liquid directly wets solid surfaces without spatial confinement. This unique feature enables spatial patterning via controlling the insertion of protruding structures into the gel.

In one example application, this process was used with protruding micro/nano structures (e.g., microneedle electrodes for electrophysiological recording), which are used extensively as probes for electrophysiological recording, biochemical sensing, electrical stimulation, and drug delivery. In this respect, control of the recording regions is typically necessary to target the regions of interest with such micro/nano structures. In some aspects, accurate control of recording regions can be realized by conformal coverage of a thin insulation layer (e.g., parylene C, a type of dielectric polymer) on the microneedles except for the tips, which can serve as the exposed conductive regions for electrophysiological recording.

In some aspects, by partially inserting microneedles in liquid etchant-filled agarose gel, controlled chemical etching of thin films on the tips of the microneedles is achieved for patterning the exposed area of the microelectrode tip. An array of polymer (e.g., polyimide) microneedles are used and metallization of the microneedles can be realized by conformally coating the microneedles with a thin layer of metal (e.g., Cr/Au, approximately 10 nm/150 nm in thickness) by sputtering. Subsequently, a conformal coating of insulation material, such as parylene C (e.g., approximately 3 μm in thickness), is applied to the microneedles, followed by patterned deposition of a hard metal mask (e.g., approximately Cu, 200 nm in thickness) through a shadow mask (see, e.g., view A of FIG. 6) that prevents Cu deposition outside of the microneedle surfaces.

Views A to I of FIG. 8 show schematic illustrations related to example implementations with related experimental data according to aspects of this disclosure. In particular, FIG. 8 shows views related to ex vivo recordings of intramuscular and surface EMG from the buccal mass of Aplysia using example fabricated systems and planar MEA devices according to this disclosure. View A of FIG. 8 shows a schematic illustration of a retraction movement cycle in the buccal mass. Views B and C of FIG. 8 show an optical image of a buccal mass with an example fabricated system (View B) and a graph demonstrating corresponding dimensional changes during a retraction movement cycle (View C).

In view B of FIG. 8, the investigated, example fabricated system included 8-channels with varying microneedle lengths (0.4 to 1.5 mm). The system was attached on an isolated buccal mass longitudinally with the microneedle electrodes inserted into the muscle tissues. The application of a hydrogel-based bioadhesive between the example system and the buccal mass surface ensured stable device adhesion to the buccal mass.

In some aspects, device adhesion to the buccal mass. During a typical cycle of retraction movement, there exist three distinct stages: contracting, relaxing, and resting (view A of FIG. 8). In the first stage (stage I), the anterior buccal mass retracts with the I1/I3 muscles contracting and the I2 muscle relaxing. As a result, the diameter at the anterior end (R) decreases, while the length from the mouth to the esophagus (L) increases (View C). In stage II, the anterior buccal mass expands as I1/I3 and I2 muscles relax, causing opposite changes in L and R. In stage III, the buccal mass is in a resting state with both the I1/I3 and I2 muscle groups relaxed. As shown in view F, the recorded intramuscular EMG signals from the electrodes 1 to 4 of the example system and the corresponding power spectral density graph demonstrated significantly higher amplitude than those recorded from electrodes 5 to 8 in stage I, when the I1/I3 muscles was contracting and I2 muscle was relaxing. The amplitude of the EMG signals decreased in stage II and stage III, where both the I1/I3 and I2 muscles were relaxed. The power spectrum analysis in view H of FIG. 8 demonstrated peak amplitudes centered around approximately 5 to 20 Hz in stage I, characteristic of intramuscular EMG in Aplysia.

In view C, R represents the diameter of the buccal mass at the anterior, and L represents the length from the mouth to the esophagus. View D shows confocal microscopic images showing the microneedle electrodes inserted into the I1/I3 and I2 muscle groups of the buccal mass. View E shows an optical image with identification of each microneedle electrode inserted into the buccal mass. Views F and G show graphs depicting intramuscular EMG and surface EMG signals recorded by the example fabricated system (view F) and the planar MEA (view G). In view F, the term, “MN” means microneedle electrodes and view G the term “P” means planar electrodes. Views H and I show power spectra of the intramuscular EMG and surface EMG signals averaged across all recording channels from (view H) the example fabricated system and (view I) the planar MEA. The scale bars in views B and E are 5 mm and 2 mm in view D.

In FIG. 8, it was observed that large stretchability combined with relatively high modulus of the microneedles made it so that the investigated, fabricated system is configured to record electrophysiological signals within dynamically moving tissues, such as intramuscular EMG. In some aspects, the buccal mass of the marine mollusk Aplysia californica represented a model that contains a dense assembly of distinct muscle groups responsible for feeding, biting, and swallowing. It was therefore demonstrated that the investigated, example fabricated system was able to sense intramuscular EMGs from an isolated, dynamic buccal mass of Aplysia.

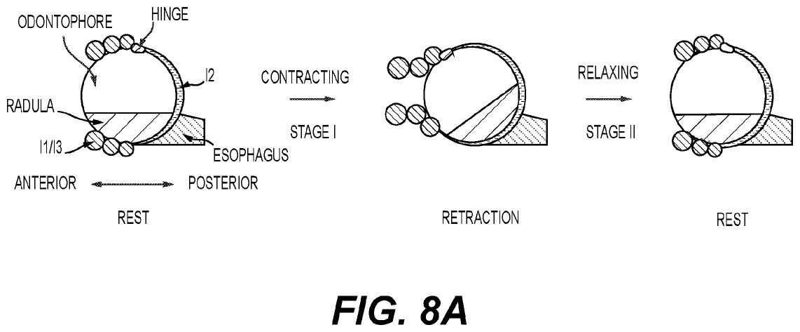

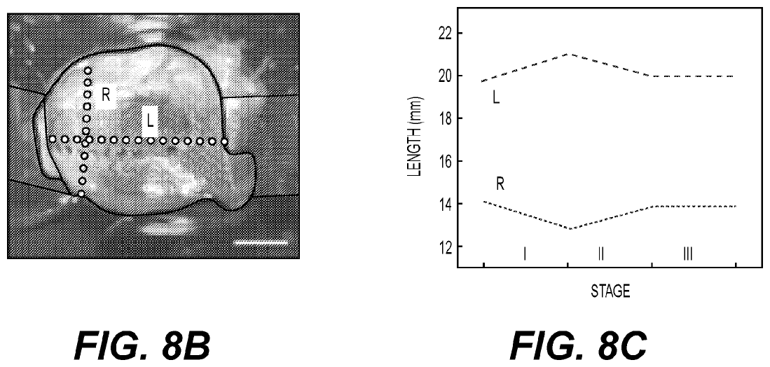

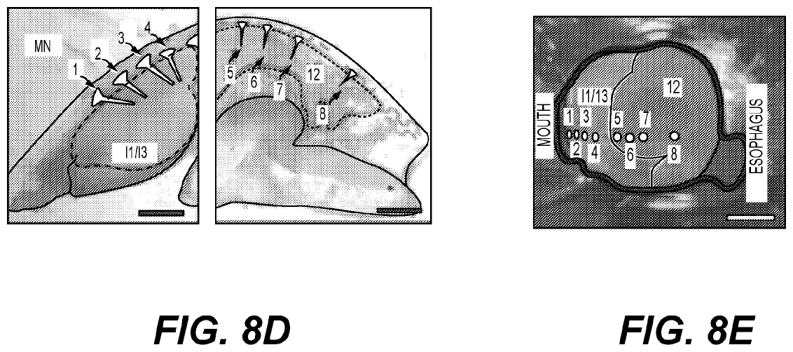

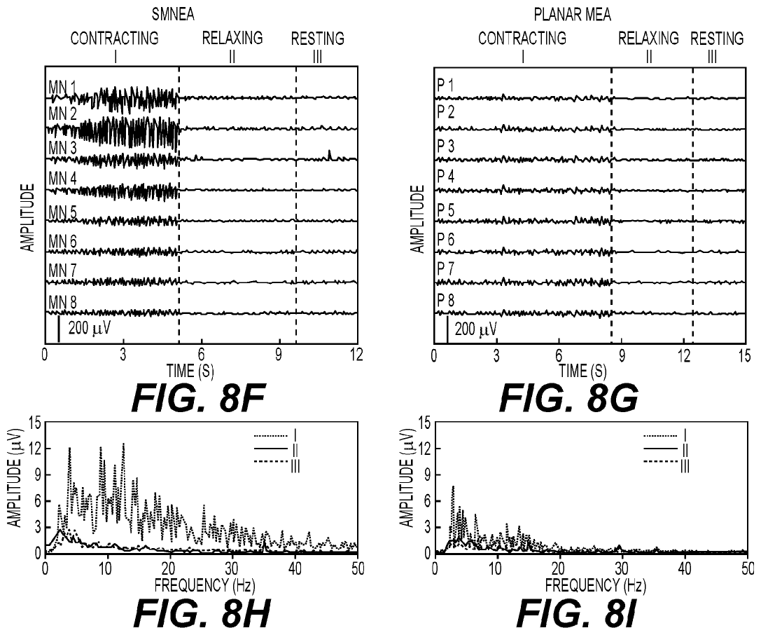

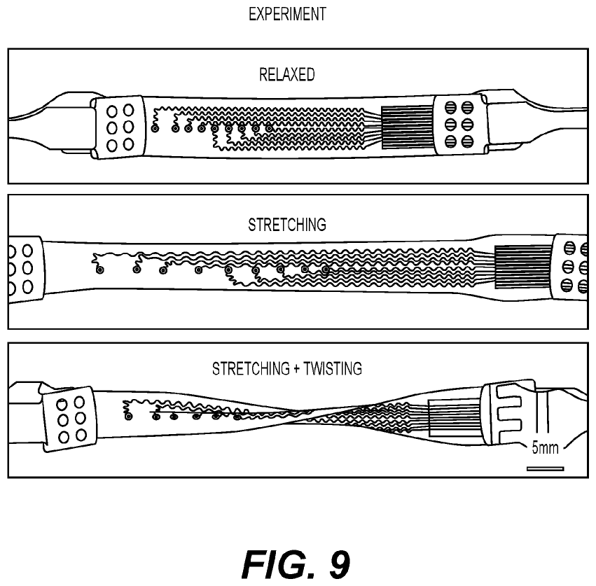

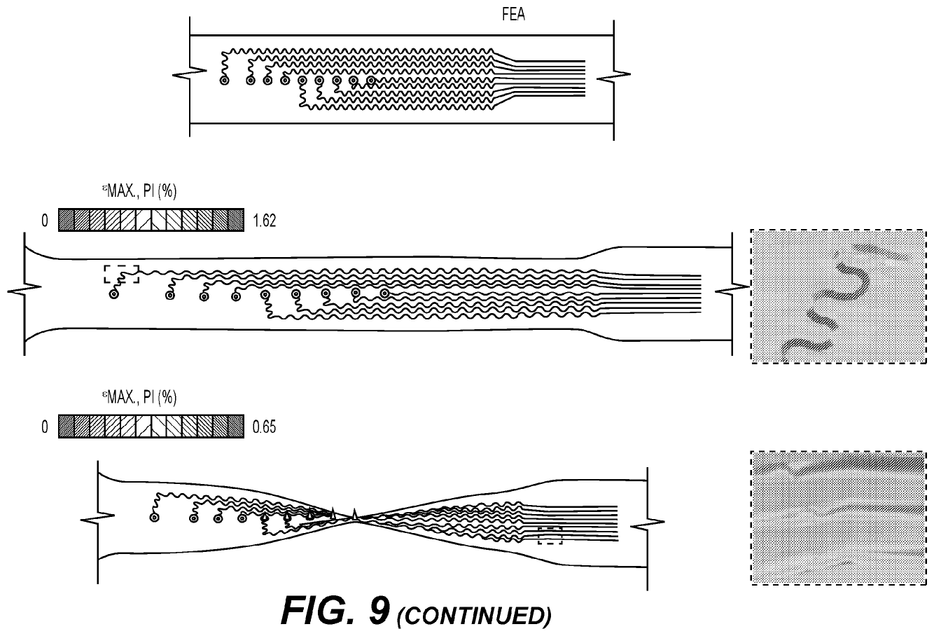

FIG. 9 show schematic illustrations related to example implementations with related experimental data according to aspects of this disclosure. View A shows side-view optical images and the corresponding finite element analysis (FEA) results showing the maximum principal strain distribution in the PI layer of the example fabricated system at relaxed state, under stretching and a combination of stretching and twisting. Insets show the locations of the maximum value of the maximum principal strain distributions.

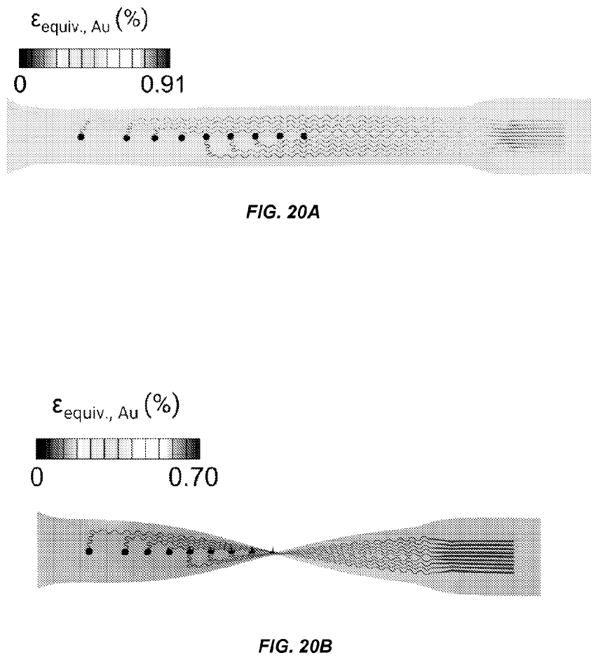

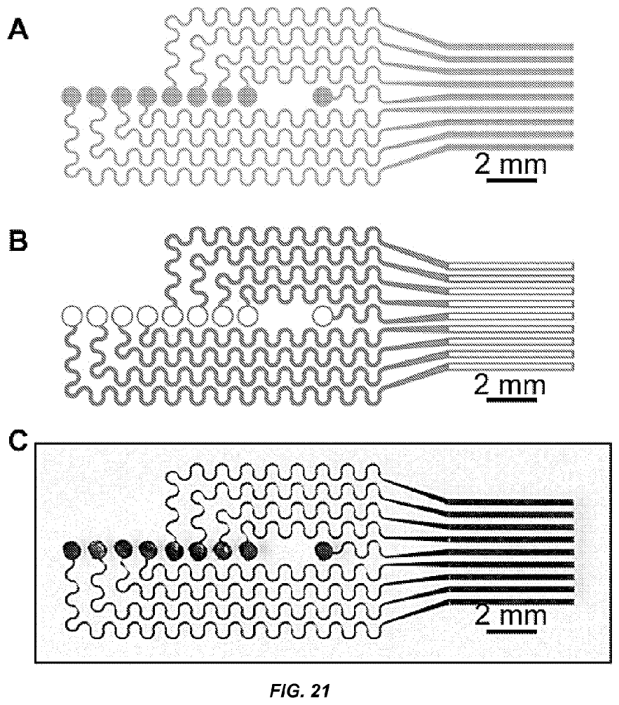

The Au/PI serpentine interconnects bonded to the silicone substrate were observed to give rise to mechanical stretchability of the example fabricated system (e.g., system 100). FIG. 9 shows an example fabricated system under stretching (66% tensile strain), and a combination of stretching (30% tensile strain) and twisting (180° rotation) deformations. Finite element analysis (FEA) can provide quantitative strain distributions in the constituent materials in the example fabricated system. For stretching, the maximum principal strain in the PI layer appears near the serpentine edges, reaching 1.62% for 66% tensile strain of the example fabricated system. The strain level in the PI layer for a combination of 30% tensile strain and 180° twisting is approximately 0.65%. The equivalent strain of the Au layer for the deformations in FIG. 9 appear in FIGS. 20A to 20B.

In FIG. 9 it was observed that stretching of a single row of microneedle electrodes caused increased distances between them and straightening of the interconnects. It was also observed that electrode impedance remained mostly unchanged below approximately 40%, and a significant increase in impedance was observed from approximately 50-70% strain. In this strain range, the maximum value of the maximum principal strain in the PI layer of the interconnects was observed approaching its fracture limit (1.76%), causing breaking of the serpentine interconnects. The strain in the Au layer was observed as remaining within its fracture limit of approximately 1-2%. It was observed in turn that one limiting factor of the stretchability was the fracture of the PI layer. In some aspects, the measured variations in the stretchability of different electrodes likely originated from imperfections of the PI film fabrication and patterning.

Views A to C of FIG. 10 show schematic illustrations related to example implementations with related experimental data according to aspects of this disclosure. View A shows side-view optical images of a fabricated system (e.g., system 100) under uniaxial stretching up to 100%. View B of FIG. 10 is a graph that shows impedance of the electrodes in the fabricated system as a function of the tensile strain applied. View A of FIG. 10 shows side-view optical images of examples of the fabricated system under uniaxial stretching up to 100%, whereby the fabricated system includes PtB coating at the tips under uniaxial stretching with the fabricated system immersed in PBS for electrical impedance measurement. View B of FIG. 10 is a graphical comparison of impedance of the electrodes in the example fabricated system as a function of tensile strain applied. Scale bars are 5 mm in view A left and 2 mm in view A right. View C of FIG. 10 is a graphical comparison of the example fabricated system with previously reported flexible or stretchable microneedle electrode arrays in the microneedle modulus and the device stretchability.

In this disclosure, FEA simulations for the performance of example systems were conducted by the commercial software ABAQUS. Eight-node 3D stress solid elements (C3D8R) were used to mesh the structure with fine element size (approximately 100,000 elements) smaller than the serpentine width to ensure accuracy. The stretching and twisting were applied by corresponding translation or rotation displacements on the two ends of the sample. The materials were described by different constitutive models including a linear elastic model for PI and Au, and a Mooney-Rivlin hyper-elastic model for Ecoflex. The Young's moduli of PI and Au are E_{PI}=6.63 GPa, E_{Au}=79 GPa, respectively. The Poisson's ratios are v_{PI}=0.4, v_{A}=0.44, respectively. The yield stress of the Au was 546 MPa and corresponding strain was 0.7% for Au thin films. The Mooney-Rivlin strain potential of Ecoflex is governed by:

$$U = {C}_{10} ( {I}_{1} - 3 ) + {C}_{01} ( {I}_{2} - 3 ) + {{D}_{1}^{- 1} ( {J}^{el} - 1 )}^{2}$$

where C₁₀=0.008054 MPa, C₀₁=0.002013 MPa, and D₁=2.0 MPa⁻¹. I₁ and I₂ are the first and second invariants of the deviatoric strain tensor and J^{el} is the elastic volume ratio representing the thermal expansion.

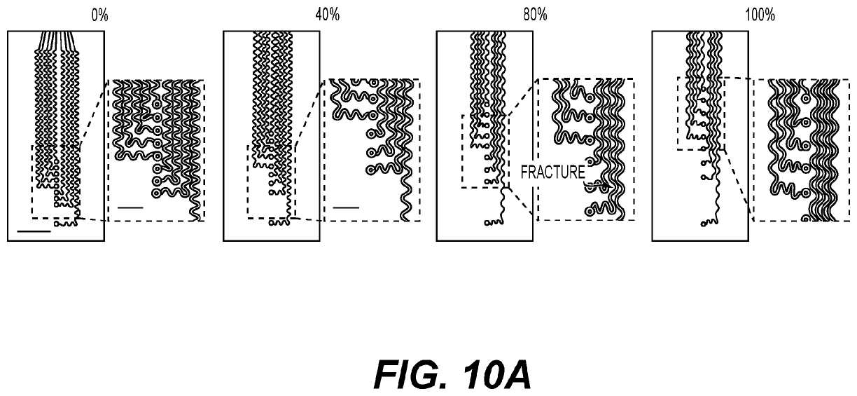

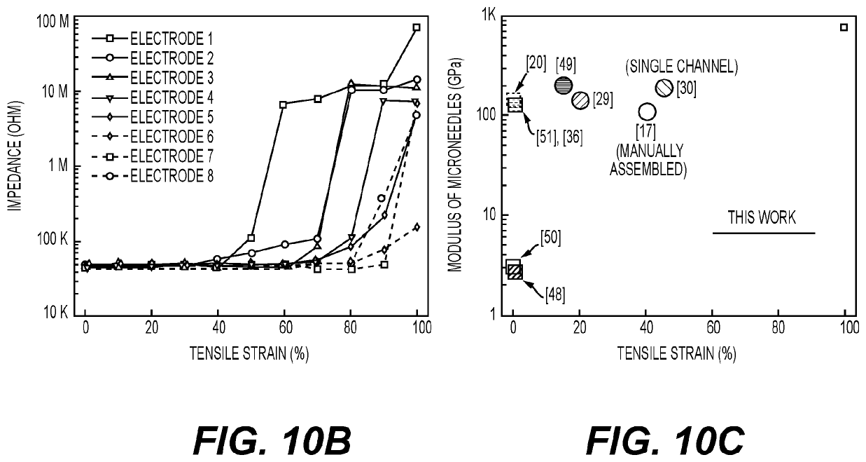

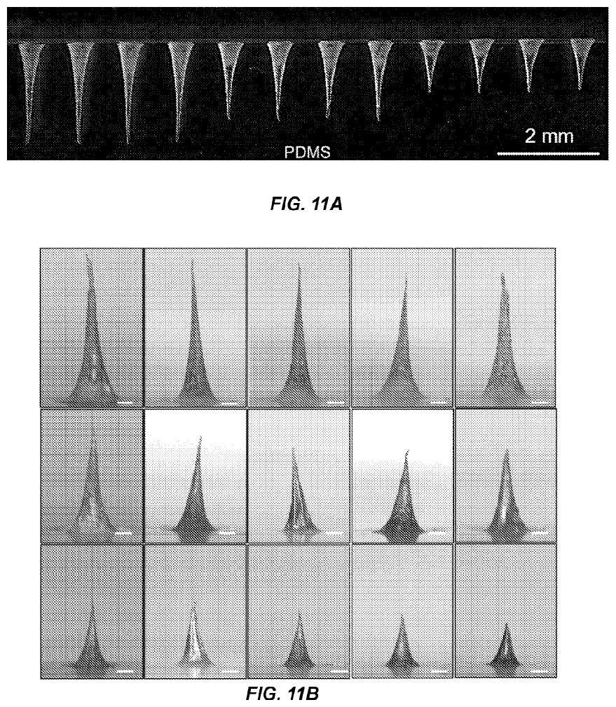

In FIGS. 11A and 11B, example optical images of example implementations are shown. In some aspects, the fabrication of systems 100 of this disclosure can include incorporating a hybrid process, such as in FIGS. 11A and 11B, that combines replica molding and planar microfabrication. In this respect, the process can begin with the creation of conical cavities by laser ablation of polydimethylsiloxane (PDMS) using laser cutter (e.g., a low-cost CO2 laser cutter as in FIG. 11A). FIG. 11A shows a side-view optical image of a PDMS mold with conical cavities fabricated by laser micromachining with a scale bar of 2 mm. In some aspects, controlling laser power, ablation pattern, and focus can yield conical cavities with various base diameters and depths, as in FIG. 11B which shows a side-view of optical images of example fabricated microneedles. In particular, the images of FIG. 11B show PI 2610 microneedles fabricated by replica molding with scale bars of 200 μm.

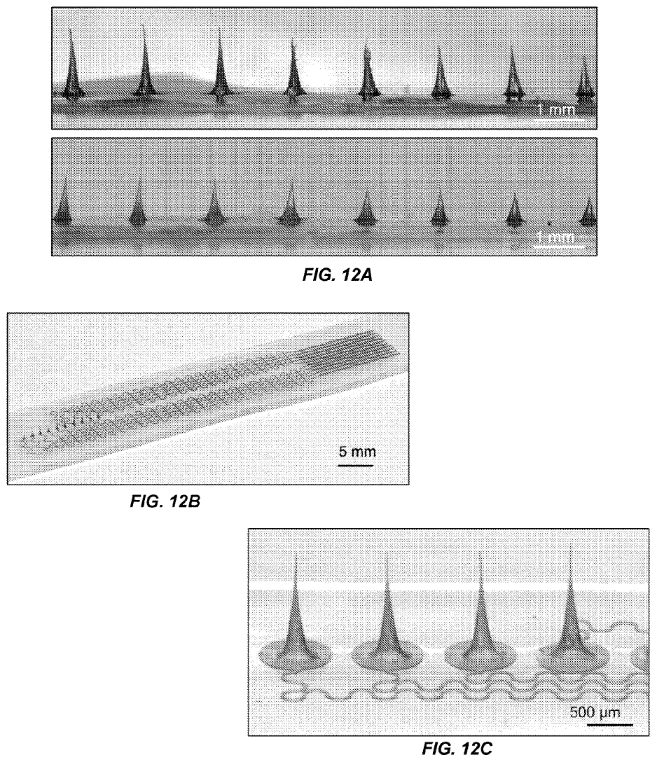

FIG. 12A shows side-view optical images of microneedles (e.g., made of PI 2610) with different base diameters and heights on one example substrate (e.g., a PI substate) at scale bars of 1 mm. In the example shown in FIG. 12A, the PDMS with cavities serves as a mold for replicating microneedle structures. The replica molding process can start with surface treatment of the PDMS mold using atmospheric plasma, followed by deposition of PI 2610 into the cavities, spin coating, and a multi-step curing process. The result of this example is an array of PI microneedles with desired geometries inside the PDMS cavities connected by a thin film of PI on the top surface, as in FIG. 12A. Microneedles of this example can have base diameters ranging from 300 to 800 μm, lengths ranging from 500 to 2000 μm, and tip diameters of approximately 10-30 μm.

FIG. 12B shows an optical image of example microneedles (e.g., PI 2610) with PI interconnects on a silicone substrate at an angle-view of a stretchable PI 2610 microneedle array at a scale bar of 5 mm. FIG. 12C shows an optical image of example microneedles (e.g., PI 2610) with PI interconnects on a silicone substrate at an angle-view close-up image of the PI microneedles at scale bars of 500 μm. In this example, the flat PI surface due to spin coating can allow lithography-based microfabrication processes on the PI thin film. Patterned etching of the PI thin film here can create an outline (30 μm in width) that defines the serpentine interconnects and edges of the microneedle bases (FIGS. 12B to 12C). Subsequent deposition of a Ti/SiO₂ layer in this example on the PI surface can allow covalent surface reactions between the SiO₂ and an oxygen-plasma-treated silicone elastomer film (Ecoflex; approximately 200 μm in thickness), enabling the transfer of the microneedle arrays from the PDMS mold to the silicone elastomer. In this example, deposition of a thin Cr/Au layer (10 nm/150 nm in thickness) can metallize the microneedles and serpentine filaments, followed by the removal of the excess PI film outside of the outlines of the microneedles and serpentine filaments.

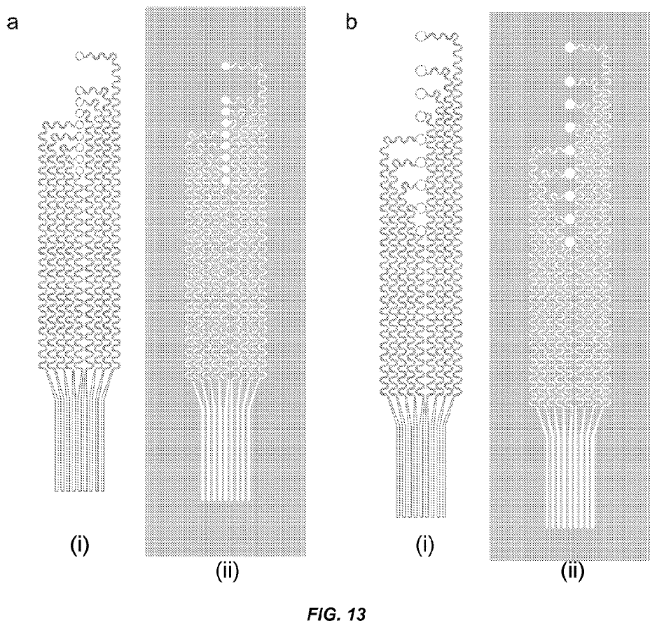

FIG. 13 shows layouts of an example PI pattern (subviews (i)) and an example sacrificial layer pattern (subviews (ii)) for a row of microneedle electrodes with view A at 1 mm spacing and view B at 2 mm spacing. The layouts of the PI pattern and the sacrificial layer pattern shown in FIG. 13 relate to those of view B of FIG. 7 and FIG. 14 which shows layouts of an example PI pattern (view A) and an example sacrificial layer pattern (view B) for an example array according to aspects of this disclosure.

FIG. 15A shows SEM images of a microneedle tip (View A) before and (View B) after removing the Cu hard mask which includes a scale bar of 10 μm. FIG. 15B shows an optical image of a Cu-coated microneedle tip immersed in the custom gel etchant with minimal etchant spreading at a scale bar of 50 μm.

FIG. 16A shows a SEM image of a microneedle tip after electrochemical deposition (e.g., deposition of PtB) at the tip with a scale bar 30 μm. FIG. 16B shows electrode impedance spectra and average electrode impedance at 1-kHz scanning frequency before and after electrochemical deposition of PtB at the microneedle tip in 0.1 M PBS. In FIG. 16B, the error bars correspond to the calculated standard deviation from 12 electrode measurements. FIG. 16C shows impedance of the electrode with the microneedle tip at 1 kHz under cyclic insertion into agarose gel.

FIG. 17A shows a SEM image of an example microneedle with the tip exposed with a scale bar of 200 μm. FIG. 17B shows an optical image of an example patterned gel etchant for etching a hard mask (e.g., Cu hard mask) on a microneedle in an array of microneedles with varying heights with a scale bar of 300 μm.

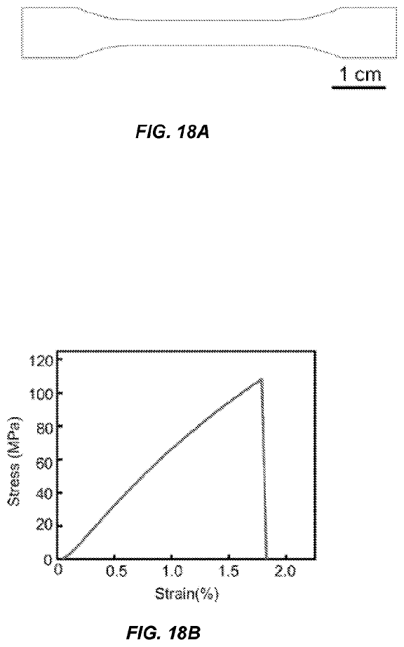

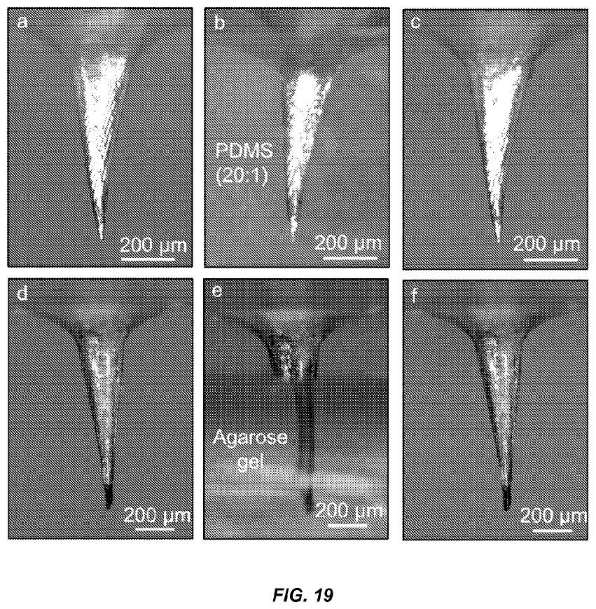

FIG. 18A shows an example pattern of thin film for tensile testing according to an example. FIG. 18B shows a stress-strain curve of the thin film example of FIG. 18A from uniaxial tensile testing. In some aspects, core material of the microneedle is PI 2610, which is a thermoplastic polymer with high mechanical modulus and thermal resistance. A tensile test on a flat PI 2610 film (30 μm in thickness; pattern appears in FIG. 18A) was prepared by a spin coating and curing process yields a stress-strain curve shown in FIG. 18B. It was observed that the PI 2610 film exhibited a linear stress-strain relation with a fitted Young's modulus of 6.63 GPa before fracturing at 1.76% tensile strain.

This relatively high modulus compared to that of most other polymeric microneedles facilitated the insertion of the microneedles into target tissues without bending of the microneedle tips. The microneedles were also observed to exhibit robust mechanical and electrical properties over repeated insertions into soft materials. For example, PI microneedles were observed to penetrate PDMS (10:1 mixing ratio; Young's modulus of 2.8 MPa) without visible tip bending. Au-coated microneedles exhibited no visible tip bending (see views A to C of FIG. 21) and slight (40%) increase in the electrical impedance after 1000 times of insertion into PDMS (20:1 mixing ratio; Young's modulus of 0.6 MPa).

Similarly, PtB-coated microneedles show a 4.6% increase in the impedance without visible tip bending or PtB delamination after 1000 times of insertion into agarose gel (see, e.g., views D to F of FIG. 19). In this respect, views A to C of FIG. 19 show schematic illustrations related to example implementations of Au-coated microneedle insertion tests according to aspects of this disclosure, where view A shows before insertion, view B shows first inserted into PDMS (20:1 mixing ratio), and view C shows after the 1000th insertion into PDMS (20:1 mixing ratio). Views D to F of FIG. 19 show schematic illustrations related to example implementations of an Au-coated microneedle with tip coated with PtB according to aspects of this disclosure, where view D shows before insertion, view E shows first inserted into agarose gel, and view F shows after the 1000th insertion into agarose gel.

In some aspects, the low-cost, scalable microneedle fabrication process disclosed herein can be based on laser micromachining and molding (e.g., in conjunction with backside lithographic patterning). In some aspects, this fabrication process is configured for the formation of individually addressable, high-modulus microneedle arrays connected to interconnects (e.g., serpentine-shaped interconnects). In some aspects, covalent bonding between microneedles and interconnects to a stretchable substrate yields high stretchability. In some aspects, metallization and a gel-based chemical etching technique applied to the microneedles of this disclosure can yield microneedle electrode arrays with controllable exposed areas.

In some aspects, a basic structure of the example fabricated systems disclosed herein can include arrays of microneedles connected (e.g., individually) by serpentine interconnects. In some aspects, the microneedles and interconnects can be covalently bonded to a stretchable substrate (e.g., a silicone elastomer, such as Ecoflex 00-30). In some aspects, fabrication of one or more example systems provides a hybrid strategy combining replica molding and planar microfabrication. One exemplary process starts with the creation of conical cavities by laser ablation of polydimethylsiloxane (PDMS) using a low-cost CO₂ laser cutter so as to control laser power, ablation pattern, and focus can yield conical cavities with various base diameters and depths. In some aspects, the PDMS with cavities can serve as a mold for replicating microneedle structures. In some aspects, the replica molding process can start with surface treatment of the PDMS mold using atmospheric plasma, followed by deposition of polyimide (e.g., PI 2610) into the cavities, spin coating, and a multi-step curing process. In. turn, an array of microneedles with desired geometries is produced inside the PDMS cavities connected by a thin film of polyimide on the top surface.

In some aspects, the surface of the substrate that includes PI (e.g., due to spin coating) can be configured to allow lithography-based microfabrication processes on the PI thin film. In some aspects, patterned etching of the PI thin film creates an outline (e.g., approximately 30 μm in width) that defines the interconnects and edges of the microneedle bases. Subsequent deposition of a layer (e.g., Ti/SiO₂ layer) on the PI surface allows covalent surface reactions between the SiO₂ and an oxygen-plasma-treated silicone elastomer film (e.g., Ecoflex; approximately 200 μm in thickness), enabling the transfer of the microneedle arrays from the PDMS mold to the substrate (e.g., silicone elastomer). Deposition of a relatively thin Cr/Au layer (e.g., 10 nm/150 nm in thickness) metallizes the microneedles and filaments of the interconnects, followed by the removal of the excess PI film outside of the outlines of the microneedles and interconnect filaments. In some aspects, the herein disclosed example hybrid fabrication strategy can allow for the formation of highly customized microneedle geometries and array layouts using low-cost laser ablation and standard microfabrication processes. In. some examples, relatively large arrays of stretchable microneedle electrodes with varying microneedle lengths can be created using this hybrid fabrication approach.

In some aspects, fabrication of the stretchable microneedles of those example fabricated systems of this disclosure began with curing (approximately 80° C. for approximately 2 hrs) a sheet of polydimethylsiloxane (PDMS, Dow Sylgard 184) (10:1 mixing ratio, 2 mm in thickness), followed by ultraviolet (UV) ozone treatment (270 sec) of the cured PDMS surface and bonding of the treated surface to a glass slide. Laser ablation of the PDMS was performed using a low-cost CO₂ laser cutter (Universal Laser System PLS 4.75) which created conical cavities in the PDMS, which served as the molds for the replica molding of the example microneedles. In some aspect, circular ablation patterns (repeated twice) were used with approximately 17% power, approximately 15% speed, and approximately-4.5 mm Z axis offset. Preparation of the PDMS molds for microneedle molding included cleaning of the PDMS molds with sonication (approximately 5 min) in isopropyl alcohol (IPA), immersion (approximately 2 min) in 3-Aminopropyltriethoxysilane (approximately 0.01% in IPA solution), drying, and corona treatment (Electro-Technic Products) of the surface for approximately 30 sec. Initial deposition of liquid polyimide 2610 precursors (HD Microsystems) onto the mold filled the cavities, assisted by vacuum degassing (30 min). Deposition of additional PI 2610 precursors onto the molds followed by spin coating (approximately 600 rpm for approximately 30 s) and a multi-step curing process (approximately 35° C. for approximately 1 hr, approximately 50° C. for approximately 1 hr, approximately 80° C. for approximately 1 hr, approximately 110° C. for approximately 1 hr, approximately 230° C. for approximately 4 hrs) in an oven formed a thin PI film.

In some aspects, patterning of the thin PI film formed in the replica molding process started with deposition and lithographically patterned etching of a Cu hard mask (e.g., approximately 200 nm in thickness) on the cured PI film. The hard mask transferred the layouts of circular shaped microneedle bases and serpentine interconnects to the PI layer via oxygen plasma etching of the PI to form patterned gaps (e.g., approximately 30 μm in width) that separate the microneedle bases and serpentine interconnects and the rest of the PI film. After the removal of the hard mask in Cu etchant, a lithographic step defined a sacrificial photoresist layer (AZ 5214) on the PI film outside the microneedle bases and serpentine filaments, followed by a sputtering deposition of Ti/SiO₂ (approximately 10 nm/50 nm in thickness). Separately, a silicone film (Ecoflex 0030, Smooth-On, Inc.; approximately 200 μm in thickness) spin coated and cured on a polyvinyl alcohol (PVA)-coated PI film (approximately 50 μm in thickness) served as the stretchable substrate for the example fabricated system. Transfer of the PI from the PDMS mold to the Ecoflex substrate began with UV ozone treatment of the Ecoflex and corona treatment of the PI surface to create hydroxyl groups on the surfaces, followed by mechanical bonding with them and heating (approximately 70° C. for approximately 10 min). Immersion of the bonded PI in IPA facilitated the peeling of the PI from the PDMS mold. After successful transfer of the PI to an Ecoflex substrate, sputter deposition of a layer of Cr/Au (approximately 10 nm/150 nm in thickness) metallized the microneedles and the serpentine filaments. Immersion of the sample in acetone undercut the sacrificial photoresist layer. Peeling of the PI film outside of the microneedle bases and serpentine filaments from the Ecoflex substrate completed the process, facilitated by the patterned sacrificial layer between the PI and the Ti/SiO₂ bonding layer.

In some aspects, the process of controlled microneedle conductive tip exposure can begin with conformal coating (Specialty Coating Systems, PDS 2010) of an insulating parylene C (approximately 5 μm in thickness) on the SMNEA. Then a thin Cu layer (approximately 200 nm in thickness) sputter coated through a shadow mask covered the sidewalls of the microneedles, serving as a hard mask for tip exposure. Separately, a gel etchant was prepared by soaking an agarose gel (approximately 0.6 g in 100 mL deionized water; Sigma-Aldrich) in diluted FeCl₃/HCl (approximately 1:20 dilution; Sigma-Aldrich) for approximately 8 hrs. Brief (approximately 5 sec) insertion of the Cu-coated microneedle tips in the gel etchant removed the Cu hard mask at the tips. Control of the microneedle insertion length relied on a high-precision linear stage (VT 80, Physik Instrumente) that the microneedles were attached to with side-view microscopic imaging (Keyence). Following the selective removal of the Cu hard mask at the microneedle tips, an oxygen plasma etching removed all the exposed parylene C not covered by the hard mask. Removal of the hard mask in liquid Cu etchant completed the patterning of the insulation coating. An additional coating of Ecoflex (approximately 200 μm in thickness) on the example system can provide encapsulation of the serpentine interconnects and the microneedle bases after electrical connection between the contact pads on the Au-coated PI and anisotropic conductive film (ACF) cables.

In some aspects, impedance measurements and electrochemical deposition of this example relied on a potentiostat and impedance analyzer (PalmSens4) with a Pt electrode as the counter electrode and Ag/AgCl as the reference electrode. Impedance measurements were conducted in PBS (pH 7.4) at frequencies from approximately 5 to 10 kHz. Electrochemical deposition of PtB was conducted in a mixture of approximately 0.6 g chloroplatinic acid (PtCl₆H₂; Sigma-Aldrich), approximately 5 mg lead acetate (Pb(C₂H₃O₂)₂; Sigma-Aldrich), and approximately 20 mL DI water using chronoamperometry (approximately −0.1 V for approximately 20 s).

In some aspects, a PI 2610 film (approximately 30 μm in thickness) was prepared using similar procedures as those used in the fabrication of other example fabricated systems of this disclosure which served as a sample for mechanical characterization. Uniaxial tensile testing (Mark-10) of a dog-bone shaped specimen patterned by laser ablation yielded force vs. displacement data. The Young's modulus was calculated from a linear fitting of the stress-strain curve. In some aspects, insertion tests of this disclosure relied on the use of a liner stage (VT 80, Physik Instrumente) to insert microneedles into PDMS (approximately 20:1 mixing ratio) or agarose gel, with an insertion of approximately 0.8 mm. Impedance measurements of the microneedle electrodes were performed after the 1st, 5th, 10th, 20th, 50th, 100th, and the 1000th insertion. In some aspects, stretchability of the example fabricated systems was evaluated by attaching one end of the example fabricated system to a motorized force tester (Mark-10) and keeping the other end fixed with the microneedle electrodes immersed in PBS (pH 7.4) allowed impedance measurements of the electrodes with different tensile strains. Impedance measurements were taken for increments of 10% elongation.

Views A to C of FIG. 21 show schematic illustrations related to example implementations of from an octopus arm according to aspects of this disclosure. Specifically, views A to C of FIG. 21 show an example fabricated microelectrode array for measuring surface EMG signals from an example octopus arm. View A of FIG. 21 shows an example layout of the example PI pattern. View B of FIG. 21 shows an example the sacrificial layer pattern for a planar microelectrode array. View C of FIG. 21 shows an example optical image of a fabricated planar microelectrode array.

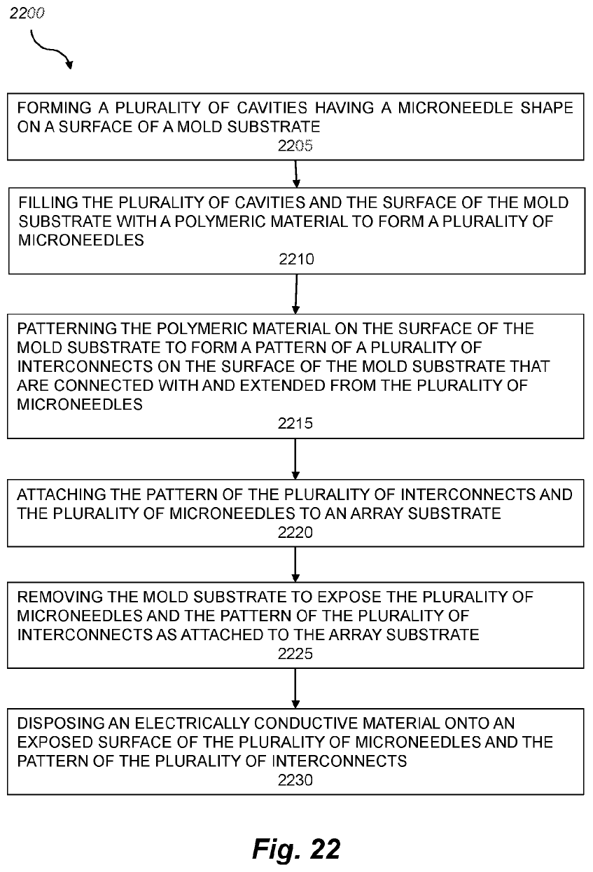

In some examples, method 2200 is disclosed related to making a microneedle electrode array and is described in the flowchart of FIG. 22. Step 2205 of method 2200 can include forming a plurality of cavities having a microneedle shape on a surface of a mold substrate. Step 2210 of method 2200 can include filling the plurality of cavities and the surface of the mold substrate with a polymeric material to form a plurality of microneedles. Step 2215 of method 2200 can include patterning the polymeric material on the surface of the mold substrate to form a pattern of a plurality of interconnects on the surface of the mold substrate that are connected with and extended from the plurality of microneedles. Step 2220 of method 2200 can include attaching the pattern of the plurality of interconnects and the plurality of microneedles to an array substrate. Step 2225 of method 2200 can include removing the mold substrate to expose the plurality of microneedles and the pattern of the plurality of interconnects as attached to the array substrate. Step 2230 of method 2200 can include disposing an electrically conductive material onto an exposed surface of the plurality of microneedles and the pattern of the plurality of interconnects.

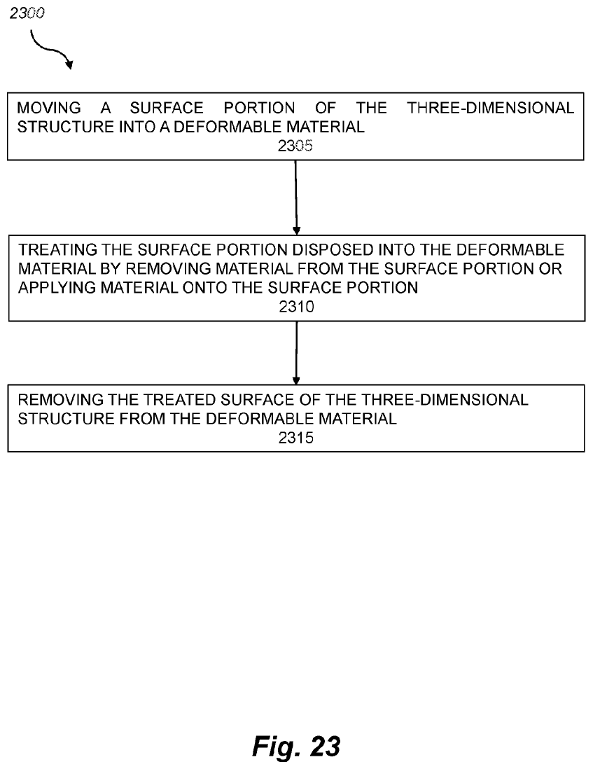

In some examples, method 2300 is disclosed related to treating a three-dimensional structure and is described in the flowchart of FIG. 23. Step 2305 of method 2300 can include moving a surface portion of the three-dimensional structure into a deformable material. Step 2310 of method 2300 can include treating the surface portion disposed into the deformable material by removing material from the surface portion or applying material onto the surface portion. Step 2315 of method 2300 can include removing the treated surface of the three-dimensional structure from the deformable material.

In some examples, systems, devices, and methods are disclosed for using porous, liquid-filled gels to achieve conformal contact with protruding micro/nano structures. In an example, the liquid or particles inside the liquid are contained within a gel (e.g., a porous gel). Upon partial insertion of protruding micro/nano structures into the gel, physical and/or chemical processes (e.g., material etching, nanoparticle deposition, etc.) occur at the gel-structure interfaces. Due to the liquid-absorbing properties of the gel, the physical and/or chemical processes are confined to the contact areas between the protruding structures and the gel. In some examples, the contact area can be controlled by adjusting the insertion location and depth, which can be guided by optical imaging. In some examples, using such technique, the fabrication of microneedle electrodes with patterned tips for localized electrical recording has been demonstrated, using liquid etchant-filled agarose gel. The exposed lengths on the microneedle electrodes can be controlled to be within approximately 10 μm as compared to the set lengths. In some aspects, this gel patterning method can allow for controlled tip exposure for microneedles with different lengths, readily achievable by shaping the gel etchants (e.g., into narrow cubes for the separate insertion of individual microneedles). In some aspects, controlled etching at the tips for a large number of microneedles is performed with vision-based, automated control of insertion (e.g., given the short insertion time) and/or by creating gel etchant patterns with predefined heights.

Although systems and methods have been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any element of FIGS. 1-23 may be modified, and that the foregoing discussion of certain of these embodiments does not necessarily represent a complete description of all possible embodiments. For example, one or more of the procedures, processes, or activities of systems and methods of this disclosure may include different procedures, processes, and/or activities and be performed by some different operation in some different order.

All elements claimed in any particular claim are essential to the embodiment claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

The specific configurations, choice of materials, concentrations thereof, steps in preparing, and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

What is claimed is:

1. A microneedle electrode array, comprising:
a substrate configured to stretch or move between a first state and a second state;
a plurality of microneedles extending outwardly from the substrate and comprising a base; and
a plurality of electrical interconnects extended from the plurality of microneedles.

2. The microneedle electrode array of claim 1, wherein the plurality of electrical interconnects is connected to the plurality of microneedles and both the plurality of electrical interconnects and the plurality of microneedles are bonded to the substrate.

3. The microneedle electrode array of claim 1, wherein the substrate is formed from silicone, and the microneedles and the electrical contacts are covalently bonded to the substrate.

4. The microneedle electrode array of claim 1, wherein the electrical interconnects have a serpentine pattern extending along a surface of the substrate.

5. The microneedle electrode array of claim 1, wherein each of the electrical interconnects is individually connected to a respective microneedle and an external circuit.

6. The microneedle electrode array of claim 1, wherein the microneedles are formed from a polymeric material having an electrically conductive material disposed on an outer surface.

7. The microneedle electrode array of claim 1, wherein a tip of the microneedles comprises electrochemical deposition of a coating comprising a conductive material that decreases an average electrode impedance of the microneedle electrode array to less than or equal to approximately 2.3 kΩ at 1 kHz in 0.1 M PBS.

8. The microneedle electrode array of claim 1, wherein the microneedle electrode array comprises an electrode impedance that is relatively unchanged below approximately 40% strain.

9. A method of making a microneedle electrode array, comprising:
forming a plurality of cavities having a microneedle shape on a surface of a mold substrate;
filling the plurality of cavities and the surface of the mold substrate with a polymeric material to form a plurality of microneedles;
patterning the polymeric material on the surface of the mold substrate to form a pattern of a plurality of interconnects on the surface of the mold substrate that are connected with and extended from the plurality of microneedles;
attaching the pattern of the plurality of interconnects and the plurality of microneedles to an array substrate;
removing the mold substrate to expose the plurality of microneedles and the pattern of the plurality of interconnects as attached to the array substrate; and
disposing an electrically conductive material onto an exposed surface of the plurality of microneedles and the pattern of the plurality of interconnects.

10. The method of claim 9, further comprising:
removing polymeric material in excess of the plurality of microneedles and the plurality of interconnects from the elastomeric substrate.

11. The method of claim 9, wherein before the step of attaching, the method comprises:
treating the pattern of the plurality of interconnects and the plurality of microneedles to form a chemical bond with the array substrate.

12. The method of claim 11, wherein the array substrate comprises a silicone material and the chemical bond formed with the plurality of interconnects and the plurality of microneedles is a covalent bond.

13. The method of claim 9, wherein during the step of patterning, the plurality of interconnects is formed having a serpentine pattern along the surface of the mold substrate to enable the plurality of interconnects to move and maintain contact with a respective microneedle of the plurality of microneedles with the array substrate stretching between a first state and a second state.

14. The method of claim 9, wherein the step of disposing the electrically conductive material onto the exposed surface of the plurality of microneedles comprises electrochemical deposition of a thin layer of a conductive coating layer onto the exposed surface.

15. A method for treating a three-dimensional structure, comprising:
moving a surface portion of the three-dimensional structure into a deformable material;
treating the surface portion disposed into the deformable material by removing material from the surface portion or applying material onto the surface portion; and
removing the treated surface of the three-dimensional structure from the deformable material.

16. The method of claim 15, wherein the three-dimensional structure is one or more protruding microneedles.

17. The method of claim 15, wherein the surface to be treated is an end portion of the three-dimensional structure, wherein the surface portion to be treated is defined by the depth of the end portion inserted into the deformable material.

18. The method of claim 15, wherein the deformable material is a gel, wherein the gel comprises a material disposed therein configured to deposit a material onto the surface during the step of treating.

19. The method of claim 15, wherein the step of treating the surface portion disposed into the deformable material by removing material from the surface portion or applying material onto the surface portion comprises material etching at least partially in an inserted region of the deformable material.

20. The method of claim 15, wherein the step of treating the surface portion disposed into the deformable material by removing material from the surface portion or applying material onto the surface portion comprises material deposition at least partially in an inserted region of the deformable material.
